(19) United States
(12) Patent Application Publication
(10) Pub. No.: US 2007/0156003 A1
(43) Pub. Date: Jul. 5, 2007

(54) Method for producing saturated hydrocarbon compound

(76) Inventors: Taiichi Furukawa, Tokyo (JP); Yong Wang, Tokyo (JP); Kinji Yamada, Tokyo (JP)

Correspondence Address: MERCHANT & GOULD PC, P.O. BOX 2903, MINNEAPOLIS, MN, 55402-0903, US

(21) Appl. No.: 11/635,660

(22) Filed: Dec. 8, 2006

(30) Foreign Application Priority Data
Dec. 9, 2005 (JP) 2005-356490

(51) Int. Cl. C07C 7/17

(52) U.S. Cl. 585/858

(57) ABSTRACT

A method for treating a saturated hydrocarbon compound suitable as a liquid for the immersion exposure method excelling in transmission in the deep ultraviolet region using sulfuric acid in an amount smaller than in a conventional method is provided. The above sulfuric acid washing treatment is the method for producing saturated hydrocarbon compound intended to reduce the absorbance, wherein the second sulfuric acid washing treatment step is conducted after the first sulfuric acid washing treatment step at a temperature 10° C. or more lower than the temperature of the first sulfuric acid washing treatment step. The absorbance of light with a wavelength of 193 nm of the saturated hydrocarbon compound to be treated in the above second sulfuric acid washing treatment step is 0.10 or less per 1 cm of a liquid optical path length, and the above saturated hydrocarbon compound is an alicyclic saturated hydrocarbon compound.

### BACKGROUND OF THE INVENTION

1. Field of the Invention

The present invention relates to a method for producing saturated hydrocarbon compounds used for an immersion exposure method and particularly to a method for producing saturated hydrocarbon compounds using a sulfuric acid washing treatment that can reduce the absorbance.

2. Background Art

In the manufacture of semiconductor devices and the like, a stepping or step-and-scan projection exposure device (aligner) has been used in which a pattern of a reticle (photomask) is transferred onto each shot region on a wafer coated with a photoresist through a projection optical system.

The theoretical limit of the resolution of the projection optical system provided in the projection exposure device increases as the exposure wavelength used becomes shorter and the numerical aperture of the projection optical system becomes greater. Therefore, the exposure wavelength which is the wavelength of radiation used in the projection exposure device has been reduced along with scaling down of integrated circuits year by year, and the numerical aperture of the projection optical system has been increased.

In this way, in the field of manufacturing semiconductor device and the like, the demand for scaling down of integrated circuits has been satisfied by reducing the wavelength of the exposure light source and increasing the numerical aperture. At present, a study is ongoing concerning mass production of a 1L1S (1:1 line and space) 90-nm half-pitch node using an ArF excimer laser (wavelength: 193 nm) as the exposure light source. However, it is difficult to achieve the next generation 65-nm half-pitch node or 45-nm half-pitch node using only the ArF excimer laser. Therefore, use of a light source with a shorter wavelength such as an F₂ excimer laser (wavelength: 157 nm) or an extreme ultraviolet (EUV) laser (wavelength: 13 nm) has been studied for the next generation technology. However, it is difficult to use these light sources under the present situation due to technological difficulty.

In the above exposure technology, a photoresist film is formed on the surface of the exposure target wafer, and the pattern is transferred to the photoresist film. In a conventional projection exposure device, the space in which the wafer is placed is filled with air or nitrogen having a refractive index of 1.

The limit of the resolution and the depth of focus can be theoretically increased by 1/n and n, respectively, by filling the space between the lens of the projection exposure device and the wafer with a liquid having a refractive index of n to provide an appropriate optical system. For example, when using water as the medium in the space between the lens and wafer in the ArF process, since the refractive index n of the light with a wavelength of 193 nm in water is n=1.44, an optical system with a resolution of 69.4% and a depth of focus of 144% can be theoretically designed compared to the exposure using air or nitrogen as the medium.

Such a projection exposure method in which the effective wavelength of exposure radiation is reduced to transfer a more minute pattern is called an immersion exposure method. The immersion exposure method is considered to be an essential technology for future lithography with reduced dimensions, particularly for lithography with dimensions of several ten nanometers, and a projection exposure device using the method has become publicly known (see Patent Document 1).

As the liquid provided between the lens of the projection optical system and the substrate in the immersion exposure method, use of pure water has been studied for the ArF excimer laser, and use of a fluorine-based inert liquid and the like has been studied for the F₂ excimer laser due to high transparency to light with a wavelength of 157 nm (see Patent Documents 2 and 3).

[Patent Document 1] JP-A-11-176727

[Patent Document 2] WO 99/49504

[Patent Document 3] JP-A-10-303114

The inventors of the present invention have investigated various compounds in order to provide an immersion exposure liquid which exhibits a refractive index higher than that of pure water, exhibits excellent transparency at an immersion exposure wavelength, prevents elution and dissolution of a photoresist film or its upper layer film components (particularly hydrophilic components), does not erode a lens, and reduces defects during formation of a resist pattern, and can form a pattern with a more excellent resolution and depth of focus when used for an immersion exposure method. As a result, the inventors have found that an alicyclic saturated hydrocarbon compound having a small absorbance in the deep ultraviolet region and a high refractive index is suitable as a liquid for immersion exposure methods (WO2005/114711).

However, alicyclic saturated hydrocarbon compounds synthesized by a known method or made commercially available generally contain impurities exhibiting significant absorbance in the deep ultraviolet region. For this reason, when used as the liquid for immersion exposure method, these alicyclic saturated hydrocarbon compounds may cause problems such as a decrease in throughput due to low sensitivity resulting from a small transmittance, defocusing and distortion of an optical image resulting from refractive index fluctuation caused by heat generation of the liquid when the liquid absorbs light, or poor resolution and impaired pattern profiles due to the defocusing of the optical image.

In order to solve these problems, as a method for efficiently removing aromatic rings and unsaturated hydrocarbon compounds abundantly contained in alicyclic saturated compounds, the inventors of the present invention have discovered that a method of refining these alicyclic saturated compounds by washing with sulfuric acid or by distilling after washing with sulfuric acid is suitable (for example, Japanese Patent Applications No. 2005-179583 and No. 2005-335869).

However, commercially available products of saturated hydrocarbon compounds exhibit a high absorbance at 193 nm. The reason is considered to be the presence of a slight amount of compounds having an unsaturated carbon-carbon bond or an aromatic ring and compounds having a functional group such as a carbonyl group and a hydroxyl group as impurities. If contained in a very small amount, these compounds significantly affect the transmittance due to an extremely strong absorbance at 193 nm. In order to remove the very small amount of these impurities using a conventional sulfuric acid washing method, washing must be repeated many times by using a large amount of sulfuric acid, but cannot sufficiently reduce the absorbance at 193 nm.

### SUMMARY OF THE INVENTION

An object of the present invention is to provide a method in which the absorbance of a saturated hydrocarbon compound suitable as a liquid for the immersion exposure method excelling in transmission in the deep ultraviolet region can be reduced and by which it is possible to process the saturated hydrocarbon compound using sulfuric acid in an amount smaller than in a conventional method.

As a result of extensive studies on the method for efficiently removing impurities contained in saturated hydrocarbon compounds, the inventors of the present invention have discovered that if such saturated hydrocarbon compounds, particularly those having absorbance of 0.10/cm or less at 193 nm, are washed with sulfuric acid at a low temperature, the absorbance can be further reduced. The inventors have further found that refining by washing with sulfuric acid at a low temperature can remove impurities more efficiently than refining at room temperature and can reduce the amount of sulfuric acid to be used for refining. These findings have led to the completion of the present invention.

Specifically, the present invention provides a method for producing a saturated hydrocarbon compound by a sulfuric acid washing treatment for reducing absorbance, wherein the sulfuric acid washing treatment comprises two or more sulfuric acid washing treatment steps, each conducted at a temperature differing from the other steps, at least the temperature in the last sulfuric acid washing treatment step being lower than the temperature in previous sulfuric acid washing treatment steps.

In the above method, the two or more sulfuric acid washing treatment steps comprise a first sulfuric acid washing treatment step and a second sulfuric acid washing treatment step, the second sulfuric acid washing treatment step being conducted after the first sulfuric acid washing treatment step at a temperature 10° C. or more lower than the temperature of the first sulfuric acid washing treatment step.

In the above method, the absorbance of the saturated hydrocarbon compound treated in the second sulfuric acid washing treatment step at 193 nm is 0.10 or less per 1 cm of a liquid optical path length.

The above method further comprises a distillation refining step after the sulfuric acid washing treatment and employs an alicyclic saturated hydrocarbon compound as the saturated hydrocarbon compound.

The present invention further provides a method for reducing the absorbance of a raw material saturated hydrocarbon compound of 0.10 or less per 1 cm of a liquid optical path length at 193 nm by a sulfuric acid washing treatment, wherein the temperature of the sulfuric acid washing treatment is from −40° C. to 10° C.

In the above method, the sulfuric acid washing treatment comprises two or more sulfuric acid washing treatment steps, in particular, two sulfuric acid washing treatment steps. The second sulfuric acid washing treatment step is conducted after the first sulfuric acid washing treatment step at a temperature 10° C. or more lower than the temperature of the first sulfuric acid washing treatment step, whereby the very small amount of impurities can be efficiently removed by washing using a small amount of sulfuric acid. As a result, if used as a liquid for an immersion exposure method, the saturated hydrocarbon compound improves the transmittance and increases the throughput due to an increase in sensitivity. Moreover, generation of heat by optical absorption of the saturated hydrocarbon compound can be suppressed. Problems such as defocusing and distortion of an optical image resulting from a refractive index fluctuation and deterioration of resolution and pattern profiles resulting from the distortion of the optical image can also be suppressed.

### DETAILED DESCRIPTION OF THE PREFERRED EMBODIMENTS

Saturated hydrocarbon compounds are liquid at temperatures in the range in which an apparatus used in an immersion exposure method is operated, and are alicyclic saturated hydrocarbon compounds. These saturated hydrocarbon compounds can be preferably used in an apparatus for an immersion exposure method or in an immersion exposure method in which light is exposed through a liquid filled in a space between a lens of a projection optical system and a substrate.

Compounds of the following formulas (1-1) to (1-8) can be given as examples of alicyclic saturated hydrocarbon compounds used as the immersion exposure liquid.

A compound shown by the formula (1-1): 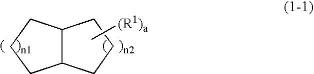
wherein R¹ represents an aliphatic hydrocarbon group having 1 to 10 carbon atoms, an alicyclic hydrocarbon group having 3 to 14 carbon atoms, a fluorine atom, or a fluorine-substituted hydrocarbon group having 1 to 3 carbon atoms, n1 and n2 individually represent integers from 1 to 3, a represents an integer from 0 to 10, when two or more R¹s exist, the R¹s may be the same or different, and two or more R¹s may be bonded to form a ring structure.

As the aliphatic hydrocarbon group having 1 to 10 carbon atoms represented by R¹, a methyl group, ethyl group, n-propyl group, and the like can be given. As examples of the ring structure formed when two or more R¹s are bonded, a cyclopentyl group, cyclohexyl group, and the like can be given. As examples of the alicyclic hydrocarbon group having 3 to 14 carbon atoms, a cyclohexyl group, norbornyl group, and the like can be given. As examples of the fluorine-substituted hydrocarbon group having 1 to 3 carbon atoms, a trifluoromethyl group, pentafluoroethyl group, and the like can be given.

As the substituents of R¹ in the formula (1-1), an aliphatic saturated hydrocarbon group having 1 to 10 carbon atoms, an alicyclic saturated hydrocarbon group having 3 to 14 carbon atoms, a fluorine atom, and a fluorine-substituted saturated hydrocarbon group having 1 to 3 carbon atoms are preferable due to the high transmittance of radiation with a wavelength of 193 nm.

Of these, the aliphatic saturated hydrocarbon group having 1 to 10 carbon atoms and the alicyclic saturated hydrocarbon group having 3 to 14 carbon atoms are particularly preferable, since the resulting product exhibits a higher refractive index and a small interaction with a resist and rarely causes defects due to elution of a water-soluble component in a resist or erosion of a lens material.

It is preferable that n1 and n2 be 1 to 3, and particularly preferably 1 or 2. It is preferable that a be 0, 1, or 2. It is particularly preferable that a be 0, since the refractive index for light with a wavelength of 193 nm increases, for example.

The alicyclic saturated hydrocarbon compounds are preferable among the preferred compounds of the formula (1-1). Among the alicyclic saturated hydrocarbon compounds, the compounds of the following formula (2-1) are particularly preferable. 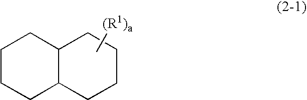
wherein R¹ and a are the same as R¹ and a in the formula (1-1).

As specific examples of preferred compounds of the formula (2-1), compounds having no substituents are preferable due to the high refractive index at 193 nm. As examples of particularly preferred compounds of the formula (2-1), cis-decalin and trans-decalin can be given. Decalin is also called decahydronaphthalene.
A compound shown by the formula (1-2): 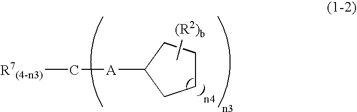
wherein A represents a single bond, a methylene group which may be substituted with an alkyl group having 1 to 10 carbon atoms, or an alkylene group having 2 to 14 carbon atoms which may be substituted with an alkyl group having 1 to 10 carbon atoms, R² is an aliphatic hydrocarbon group having 1 to 10 carbon atoms, an alicyclic hydrocarbon group having 3 to 14 carbon atoms, a fluorine atom, or a fluorine-substituted hydrocarbon group having 1 to 3 carbon- atoms, R⁷ represents a hydrogen atom, an alkyl group having 1 to 10 carbon atoms, a fluorine atom, or a fluorine-substituted alkyl group having 1 to 3 carbon atoms, n4 represents an integer from 1 to 3, b represents an integer from 0 to 6, when two or more R⁷s exist, the R⁷s may be the same or different, and two or more R⁷s may be bonded to form a ring structure, n3 represents an integer from 2 to 4, when two or more R²s exist, the R²s may be the same or different, and two or more R²s may be bonded to form a ring structure.

As examples of the methylene group which may be substituted with an alkyl group having 1 to 10 carbon atoms or an alkylene group having 2 to 14 carbon atoms which may be substituted with an alkyl group having 1 to 10 carbon atoms represented by A, an ethylene group, an n-propylene group, and the like can be given.

R² is the same as R¹ in the formula (1-1).

As the substituents of R² in the formula (1-2), an aliphatic saturated hydrocarbon group having 1 to 10 carbon atoms, an alicyclic saturated hydrocarbon group having 3 to 14 carbon atoms, a fluorine atom, and a fluorine-substituted saturated hydrocarbon group having 1 to 3 carbon atoms are preferable due to the high transmittance of radiation with a wavelength of 193 nm.

Of these, an aliphatic saturated hydrocarbon group having 1 to 10 carbon atoms and an alicyclic saturated hydrocarbon group having 3 to 14 carbon atoms are preferable for the same reason as for R¹ in the formula (1-1).

It is preferable that n3 be 2 to 4, and particularly preferably 2 or 3. It is preferable that n4 be 1 to 3, and particularly preferably 1 or 2. It is preferable that b be 0, 1, or 2. It is particularly preferable that b be 0, since the refractive index for light with a wavelength of 193 nm increases, for example.

As examples of particularly preferred compounds of the formula (1-2), 1,1,1-tricycloheptylmethane and 1,1,1-tricyclopentylmethane can be given.
A compound shown by the formula (1-3): 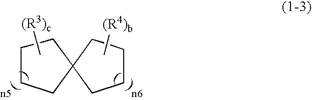
wherein R³ and R⁴ represent aliphatic hydrocarbon groups having 1 to 10 carbon atoms, alicyclic hydrocarbon groups having 3 to 14 carbon atoms, fluorine atoms, or fluorine-substituted hydrocarbon groups having 1 to 3 carbon atoms, when two or more R³s or R⁴s exist, these two or more R³s and R⁴s may be the same or different, and two or more R³s and R⁴s may respectively form a ring structure or may be bonded to form a ring structure, n5 and n6 individually represent integers from 1 to 3, and c and d represent individually integers from 0 to 8.

R³ and R⁴ are the same as R¹ in the formula (1-1).

As the substituents of R³ and R⁴ in the formula (1-3), an aliphatic saturated hydrocarbon group having 1 to 10 carbon atoms, an alicyclic saturated hydrocarbon group having 3 to 14 carbon atoms, a cyano group, a fluorine atom, and a fluorine-substituted saturated hydrocarbon group having 1 to 10 carbon atoms are preferable due to the high transmittance of radiation with a wavelength of 193 nm.

Of these substituents, an aliphatic saturated hydrocarbon group having 1 to 10 carbon atoms and an alicyclic saturated hydrocarbon group having 3 to 14 carbon atoms are preferable for the same reason as for R¹ in the formula (1-1).

It is preferable that n5 and n6 be 1 to 3, and particularly preferably 1 or 2. It is preferable that c and d be 0, 1, or 2. It is particularly preferable that both c and d be 0, since the refractive index for light with a wavelength of 193 nm increases, for example. As an example of the preferred compound of the formula (1-3), spiro[5.5]undecane can be given.
Compounds shown by the formulas (1-4) (a), (b), and (c): 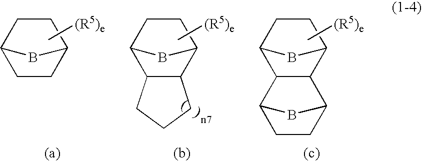
wherein B represents a methylene group or an ethylene group, R⁵ represents an aliphatic hydrocarbon group having 1 to 10 carbon atoms, an alicyclic hydrocarbon group having 3 to 14 carbon atoms, a fluorine atom, or a fluorine-substituted hydrocarbon group having 1 to 3 carbon atoms, when two or more R⁵s exist, the R⁵s may be the same or different, and two or more R⁵s may be bonded to form a ring structure, e is an integer from 0 to 10, and n7 represents an integer from 1 to 3.

R⁵ is the same as R¹ in the formula (1-1).

As the substituents of R⁵ in the formula (1-4), an aliphatic saturated hydrocarbon group having 1 to 10 carbon atoms, an alicyclic saturated hydrocarbon group having 3 to 14 carbon atoms, a fluorine atom, and a fluorine-substituted saturated hydrocarbon group having 1 to 3 carbon atoms are preferable due to the high transmittance of radiation with a wavelength of 193 nm.

Of these substituents, an aliphatic saturated hydrocarbon group having 1 to 10 carbon atoms and an alicyclic saturated hydrocarbon group having 3 to 14 carbon atoms are preferable for the same reason as for R¹ in the formula (1-1).

It is preferable that e be 0, 1, or 2. It is preferable that n7 be 1 to 3, and particularly preferably 1 or 2. It is particularly preferable that e be 0, since the refractive index for light with a wavelength of 193 nm increases, for example.

As examples of preferred compounds of the formula (1-4), compounds of the following formulas (2-2) and (2-2′) can be given. 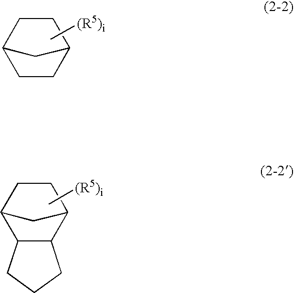
wherein R⁵ is the same as the R⁵ in the formula (1-4), and i is preferably 0, 1, or 2. It is particularly preferable that i be 0 for the same reason as for a in the formula (1-1).

As a particularly preferred example, exo-tetrahydrodicyclopentadiene can be given.
A compound shown by the formula (1-5): 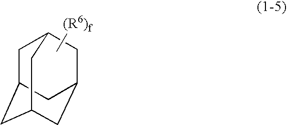
wherein R⁶ represents an aliphatic hydrocarbon group having 1 to 10 carbon atoms, an alicyclic hydrocarbon group having 3 to 14 carbon atoms, a fluorine atom, or a fluorine-substituted hydrocarbon group having 1 to 3 carbon atoms, f represents an integer from 0 to 10, and when two or more R⁶s exist, the R⁶s may be the same or different.

R⁶ is the same as R¹ in the formula (1-1).

As the substituents of R⁶ in the formula (1-5), an aliphatic saturated hydrocarbon group having 1 to 10 carbon atoms, an alicyclic saturated hydrocarbon group having 3 to 14 carbon atoms, a fluorine atom, and a fluorine-substituted saturated hydrocarbon group having 1 to 3 carbon atoms are preferable due to the high transmittance of radiation with a wavelength of 193 nm.

Of these substituents, an aliphatic saturated hydrocarbon group having 1 to 10 carbon atoms and an alicyclic saturated hydrocarbon group having 3 to 14 carbon atoms are preferable for the same reason as for R¹ in the formula (1-1).

f is preferably 1 or 2. It is preferable that the substituent be located at a bridgehead position.
A compound shown by the formula (1-6): 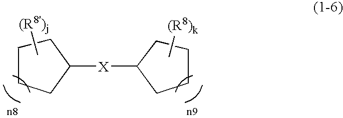
wherein R⁸ and R^{8′} represent an aliphatic hydrocarbon group having 1 to 10 carbon atoms, an alicyclic hydrocarbon group having 3 to 14 carbon atoms, a fluorine atom, or a fluorine-substituted hydrocarbon group having 1 to 3 carbon atoms, n8 and n9 individually represent integers from 1 to 3, j and k represent an integer from 0 to 6, when two or more R⁸s and R^{8′}s respectively exist, the R⁸s and R^{8′}s may be the same or different, and two or more R⁸s may be bonded to form a ring structure and two or more R^{8′}s may be bonded to form a ring structure, and X represents a single bond, a divalent aliphatic hydrocarbon group having 2 to 10 carbon atoms, or a divalent alicyclic hydrocarbon group having 3 to 14 carbon atoms.

The aliphatic hydrocarbon group having 1 to 10 carbon atoms, the alicyclic hydrocarbon group having 3 to 14 carbon atoms, and the fluorine-substituted hydrocarbon group having 1 to 3 carbon atoms represented by R⁸ and R^{8′} are the same as the aliphatic hydrocarbon group, alicyclic hydrocarbon group, and fluorine-substituted hydrocarbon group in the formula (1-1).

As the substituents of R⁸ and R^{8′} in the formula (1-6), an aliphatic saturated hydrocarbon group having 1 to 10 carbon atoms, an alicyclic saturated hydrocarbon group having 3 to 14 carbon atoms, a fluorine atom, and a fluorine-substituted saturated hydrocarbon group having 1 to 3 carbon atoms are preferable due to the high transmittance of radiation with a wavelength of 193 nm.

As examples of the divalent aliphatic hydrocarbon group having 2 to 10 carbon atoms represented by X, an ethylene group and propylene group can be given. As examples of the divalent alicyclic hydrocarbon group having 3 to 14 carbon atoms represented by X, divalent groups derived from cyclopentane, cyclohexane, and the like can be given.

As examples of the preferred compound of the formula (1-6), dicyclohexyl and dicyclopentyl can be given.
A compound shown by the formula (1-7): 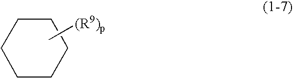
wherein R⁹ represents an alkyl group having 2 or more carbon atoms, an alicyclic hydrocarbon group having 3 or more carbon atoms, a fluorine atom, or a fluorine-substituted hydrocarbon group having 2 to 3 carbon atoms, and p represents an integer from 1 to 6, when two or more R⁹s exist, the R⁹s may be the same or different, and two or more R⁹s may be bonded to form a ring structure.

As the above-described alkyl group having two or more carbon atoms, an alkyl group having 2 to 10 carbon atoms is preferable. As examples of the alkyl group having 2 to 10 carbon atoms, a methyl group, an ethyl group, an n-propyl group, and the like can be given. As the above-described alicyclic hydrocarbon group having 3 or more carbon atoms, an alicyclic hydrocarbon group having 3 to 14 carbon atoms is preferable. As examples of the alicyclic hydrocarbon group having 3 to 14 carbon atoms, a cyclohexyl group, norbornyl group, and the like can be given. The fluorine-substituted hydrocarbon group having 2 to 3 carbon atoms is the same as the fluorine-substituted hydrocarbon group in the formula (1-1). As examples of the ring structure formed when two or more R⁹s are bonded, a cyclopentyl group, cyclohexyl group, and the like can be given.

Preferred compounds of the formula (1-7) are cyclohexyl derivatives.
A compound shown by the formula (1-8): 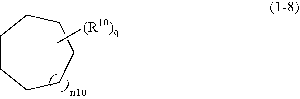
wherein R¹⁰ represents an aliphatic hydrocarbon group having 1 to 10 carbon atoms, an alicyclic hydrocarbon group having 3 to 14 carbon atoms, a fluorine atom, or a fluorine-substituted hydrocarbon group having 1 to 3 carbon atoms, n10 represents an integer from 1 to 3, and q represents an integer from 0 to 9. When two or more R¹⁰s exist, the R¹⁰s may be the same or different.

R¹⁰ is the same as R¹ in the formula (1-1). A preferred group for R¹⁰ is the same as the preferred group for R¹.

Particularly preferred compounds among the compounds of the formulas (1-1) to (1-8) are compounds having the structure of the formula (1-1) or (1-4), either unsubstituted or substituted with an aliphatic saturated hydrocarbon group having 1 to 10 carbon atoms or the alicyclic saturated hydrocarbon group having 3 to 14 carbon atoms. Unsubstituted compounds are more preferable.

As hydrocarbon compounds used as raw materials for producing saturated hydrocarbon compounds of the present invention, commercially available reagent-grade or industrial-grade products can be used. Such hydrocarbon compounds can also be synthesized from commercially available materials by a known method. The method for producing the compound of the present invention is described below by way of examples.

For example, the compound of the formula (2-1) may be produced by nuclear hydrogenation of naphthalene or a naphthalene derivative contained in carbonized oil obtained from a coke oven, a petroleum-based catalytic reformate and cracked oil obtained by fluid catalytic cracking, a naphtha cracked oil obtained as a by-product of ethylene production, or the like by catalytic hydrogenation using an appropriate catalyst.

The above catalytic reformate, cracked oil obtained by fluid catalytic cracking, or naphtha cracked oil contains naphthalene, alkylnaphthalene, and also benzene, alkylbenzene, phenanthrene, anthracene, other polycyclic aromatics and derivatives thereof, sulfur-containing compounds such as benzothiophene and derivatives thereof, and nitrogen-containing compounds such as pyridine and derivatives thereof. Naphthalene and naphthalene derivatives used as the raw material may be obtained by separation and purification from the mixture.

Naphthalene and naphthalene derivatives used to produce the compound of the formula (2-1) preferably have a low content of sulfur-containing compounds. In this case, the content of sulfur-containing compounds is preferably 100 ppm or less, and still more preferably 50 ppm or less. If the content of sulfur-containing compounds exceeds 100 ppm, the sulfur-containing compounds act as poisons during catalytic hydrogenation to hinder the progress of the nuclear hydrogenation reaction. In addition, sulfur-containing impurities originating from the sulfur-containing compounds may be mixed in the compound of the formula (2-1). The sulfur-containing compounds decrease transmittance of light with an exposure wavelength (e.g. 193 nm).

When producing cis-decalin or trans-decalin or a mixture of these as the compound of the formula (2-1), in particular, it is preferable that the naphthalene used as the raw material has a high purity. The purity of naphthalene is preferably 99.0% or more, and particularly preferably 99.9% or more. In this case, when the content of sulfur compounds as impurities is high, the above-described problem occurs. When other naphthalene derivatives, aromatic compounds, and derivatives of these are contained as impurities, these impurities produce a hydrogenated hydrocarbon compound which is difficult to separate, whereby it becomes difficult to control the purity of decalin.

As the catalytic hydrogenation catalyst, a nickel-based catalyst, a noble metal-based catalyst such as platinum, rhodium, ruthenium, iridium, or palladium, or a sulfide of cobalt-molybdenum, nickel-molybdenum, nickel-tungsten, or the like may be used. Of these, the nickel-based catalyst is preferable from the viewpoint of catalytic activity and cost.

It is preferable to use the metal catalyst in a state in which the metal catalyst is supported on an appropriate carrier. In this case, the hydrogenation rate is increased by highly dispersing the catalyst on the carrier. In particular, deterioration of the active site at a high temperature and under high pressure is prevented, and resistance to catalytic-poison is improved.

As the carrier, SiO₂, γ-Al₂O₃, Cr₂O₃, TiO₂, ZrO₂, MgO, ThO₂, diatomaceous earth, activated carbon, or the like may be suitably used.

As the catalytic hydrogenation method, a vapor phase method which does not use a solvent or a liquid phase method in which the raw material is dissolved and reacted in an appropriate solvent may be used. The vapor phase method is preferable due to low cost and high rate of reaction.

When using the vapor phase method, nickel, platinum, or the like is preferable as the catalyst. The rate of reaction increases as the amount of catalyst used increases. However, use of a large amount of catalyst is disadvantageous from the viewpoint of cost. Therefore, in order to increase the rate of reaction and complete the reaction, it is preferable to reduce the amount of catalyst and carry out the reaction at a high temperature and a high hydrogen pressure. In more detail, it is preferable to carry out the reaction using the catalyst in an amount of 0.01 to 10 parts by weight of the raw material naphthalene (naphthalene derivative) at a hydrogen pressure of 5 to 15 MPa and a reaction temperature of 100 to 400° C.

Or, the target substance may be obtained under mild conditions by removing naphthalene from tetralin (an intermediate) using nickel or a platinum or palladium-based catalyst according to the method described in the patent document (JP-A-2003-160515), for example.

In the above reaction, the reaction conversion rate is preferably 90% or more, and still more preferably 99% or more. After the reaction, it is preferable to remove unreacted raw materials and impurities such as the catalyst by appropriate purification by the following method.

The above raw materials are put into a reaction vessel. A reaction vessel made from a material from which additives such as a plasticizer are not extracted is preferably used. In addition, a material possessing high acid resistance and not invaded by alicyclic saturated hydrocarbon compounds is preferable. As preferable reaction vessels, a vessel made from a fluororesin such as polytetrafluoroethylene and a glass container can be given.

The reaction vessel is preferably washed with deionized water and a saturated hydrocarbon compound or a transparent solvent such as acetonitrile.

The raw material is then mixed with sulfuric acid for washing with sulfuric acid under prescribed temperature conditions. A commercially available sulfuric acid can be used. As sulfuric acid, fuming sulfuric acid, concentrated sulfuric acid, and sulfuric acid with an 80 wt % or more concentration can be used. A preferable concentration of the sulfuric acid is 95 wt % or more. If the concentration of sulfuric acid is less than 80 wt %, it is difficult to remove impurities by washing with the sulfuric acid. It is preferable to store sulfuric acid in a dry atmosphere or nitrogen atmosphere, because sulfuric acid absorbs moisture and deteriorates if allowed to stand in the air.

The sulfuric acid washing treatment preferably comprises two or more sulfuric acid washing treatment steps, each conducted at a temperature differing from the other steps. At least the temperature in the last sulfuric acid washing treatment step is preferably lower than the temperature in previous sulfuric acid washing treatment steps. It is particularly preferable to treat the raw material hydrocarbon in a first sulfuric acid washing treatment step and a second sulfuric acid washing treatment step. The second sulfuric acid washing treatment step is carried out at a temperature 10° C. or more lower, and preferably 20° C. or more lower, than the temperature of the first sulfulric acid washing treatment step.

The temperature of the first sulfuric acid washing treatment step is usually from 15 to 35° C., and preferably from 15 to 25° C. The temperature of the second sulfuric acid washing treatment step is from −40 to 10° C., and preferably from 0 to 5° C. The temperature difference between the first and second sulfuric acid washing treatment steps is more than 10° C.

When there is a temperature variation within the first or second sulfuric acid washing treatment step, the temperature difference between the first sulfuric acid washing treatment step and the second sulfuric acid washing treatment step is the difference between the highest temperature of the first sulfuric acid washing treatment step and the lowest temperature of the second sulfuric acid washing treatment step.

Sulfuric acid washing is carried out by mixing a raw material saturated hydrocarbon compound with sulfuric acid. As the mixing means, stirring using a stirring spring can be given. A method of increasing the stirring effect by increasing the contact surface of the raw material with sulfuric acid is preferable. For this reason, mechanical stirring using a stirring spring is preferred. If the mixing efficiency is low, removal of impurities by the sulfuric acid washing treatment is insufficient.

The sulfuric acid washing treatment is preferably carried out in an inert gas atmosphere such as nitrogen or the like. If the sulfuric acid washing treatment reaction is carried out in air, the refining efficiency is decreased due to oxidation caused by oxygen in the air and moisture absorption by the sulfuric acid.

The amount of sulfuric acid used for the sulfuric acid washing is from 25 to 100 parts by volume, preferably from 25 to 75 parts by volume, and more preferably from 25 to 50 parts by volume, per 100 parts by volume of alicyclic saturated hydrocarbons. Use of more than 100 parts by volume of sulfuric acid only increases the amount of sulfuric acid used without increasing the effect of washing, which is not preferable; if less than 25 parts by volume, the purification efficiency decreases.

The first sulfuric acid washing treatment step is carried out for 1 to 1.5 hours and the second sulfuric acid washing treatment step is carried out for 2 to 3 hours.

Each of the first and second sulfuric acid washing treatment steps may consist of either one washing run or two or more washing runs. Either the first and second sulfuric acid washing treatment steps may be carried out using fresh sulfuric acid, or the first and second sulfuric acid washing treatment steps may be continuously carried out using the same sulfuric acid.

Low-temperature sulfuric acid washing of the present invention can more efficiently remove impurities than refining by sulfuric acid washing at room temperature. Therefore, it is possible to reduce the amount of sulfuric acid used for the refining.

The types of impurities that can be removed by washing with sulfuric acid at room temperature are believed to differ from those removed at low temperature sulfuric acid washing. The reason for this is thought to be that the impurities that can be removed by sulfuric acid washing at a low temperature produce cations which are unstable at room temperature, but stable at a low temperature. On the contrary, some impurities that cannot be removed at low-temperature sulfuric acid washing are believed to be removable at room temperature. Therefore, it is possible to remove impurities more efficiently by combining room-temperature sulfuric acid washing with low-temperature sulfuric acid washing.

In the present invention, absorbance can be reduced particularly in the second sulfuric acid washing treatment, when the absorbance of a saturated hydrocarbon compound prior to the second sulfuric acid washing treatment step at a wavelength of 193 nm is 0.10 or less per 1 cm of optical path length. For this reason, it is preferable to reduce the absorbance to 0.10 or less in the first sulfuric acid washing treatment step. Moreover, if the saturated hydrocarbon raw material with an absorbance of 0.10 or less is available, it is possible to omit the first sulfuric acid washing treatment step and obtain saturated hydrocarbon compounds with sufficiently reduced absorbance by only treating such a raw material under the conditions of the second sulfuric acid washing treatment step.

The method for producing a liquid with an absorbance of 0.10/cm (an absorbance per optical path length of 1 cm) or less, in which the effect of refining by low temperature sulfuric acid washing is exhibited, can be obtained by refining a commercially available alicyclic saturated hydrocarbon compound using a combination of sulfuric acid washing at room temperature and distillation or a combination of sulfuric acid washing at room temperature, sulfuric acid washing at a low temperature and distillation.

For example, a method of washing a commercially available product three times with sulfuric acid at room temperature, followed by distillation, and a method of washing a commercially available product one time with sulfuric acid at room temperature, then one more time at a low temperature, followed by distillation can be given. In the latter case, used sulfuric acid may be replaced with fresh sulfuric acid or may be used as is, when the temperature is decreased from room temperature to a low temperature.

The absorbance of 0.10/cm per 1 cm of optical path length at a wavelength of 193 nm is converted into 97.7%/mm of transmittance in 1 mm of optical path length.

After sulfuric acid washing, it is preferable to wash with an alkali for neutralization and then wash with water, if necessary. When neutralized with an alkali, sulfonic acids produced by the reaction of concentrated sulfuric acid and aromatic hydrocarbons existing as impurities, for example, can be efficiently removed.

As the alkali used here, various alkaline metal salts such as sodium hydrogencarbonate, sodium carbonate, potassium hydroxide, sodium hydroxide, and the like are preferable. An alkaline metal salt is preferable because the alkaline metal salt dissolves only with difficulty in an alicyclic saturated hydrocarbon as an impurity.

After washing with an alkali, washing with deionized water is preferably conducted. Impurities such as metal ions contained in alkali can be removed by washing.

The above sulfuric acid treatments, neutralization with an alkali, and washing with water are carried out an appropriate number of times according to the purity, properties, and the like of the compound.

The first and second sulfuric acid washing treatments are respectively carried out preferably 2 to 10 times, and more preferably 3 to 10 times. A greater number of times of treatment increases the effect of refining, but makes the process complicated and increases the cost.

The refining efficiency can be increased by performing neutralization and washing with water for each sulfuric acid treatment. For example, neutralization and washing with water are carried out 1 to 5 times, preferably 1 to 2 times for each sulfuric acid treatment.

It is possible to perform neutralization and washing with water at the end of the sulfuric acid treatment for a suitable number of times. In this instance, neutralization and washing with water are carried out 1 to 5 times, preferably 1 to 2 times. Washing with water is preferably carried out until the pH of the liquid after washing becomes neutral.

It is possible to further reduce the transmittance by performing distillation after the above step of sulfuric acid washing. Distillation is preferably carried out under vacuum of 3 or less mmHg at a temperature of 50° C. or less.

After distillation under reduced pressure, it is preferable to return the pressure to atmospheric pressure using an appropriate inert gas and store the product as is, whereby it is possible to prevent oxidation by air during storage.

A distillation column with an appropriate number of distillation trays according to the boiling point difference of the impurities and alicyclic saturated hydrocarbons is preferably used.

The absorbance of a saturated hydrocarbon compound can be more easily reduced to 0.10/cm or less by combining sulfuric acid washing and distillation refining.

The saturated hydrocarbon compounds of the present invention obtained by the above-described refining have a high purity. In addition, because resist components, particularly volatile impurities, elute only in a small amount, optical characteristics of the liquid can be recovered with sufficient reproducibility by collecting and refining the liquid by a simple method. The reclaimed liquid can be reused.

As the method for refining the liquid for reuse, washing with water, washing with an acid (sulfuric acid washing), washing with an alkali, precision distillation, refining using an appropriate filter (packed column), filtration, the above-described refining method of the liquid according to the present invention, and a combination of these purification methods can be given. In particular, it is preferable to purify the liquid by washing with water, washing with an alkali, washing with an acid, precision distillation, or a combination of these purification methods.

The above washing with an alkali is effective for removing an acid generated in the liquid for immersion exposure upon exposure, the above washing with an acid is effective for removing a basic component in the resist eluted into the liquid for immersion exposure, and the above washing with water is effective for removing a photoacid generator and a basic additive in the resist film dissolved in the liquid for immersion exposure and eluted substances such as an acid generated upon exposure.

The refining process which comprises the concentrated sulfuric acid washing steps disclosed in the present invention is effective also in the above-mentioned recovery and purification. Since the process of the present invention can effectively remove impurities having carbon-carbon unsaturated bonds produced by decomposition of acid-labile protecting groups in a resin or by photoreaction due to the application of radiation to a liquid, the process can prevent fluctuation of the transmittance.

Precision distillation is effective for removing a low-volatile compound among the above additive, and is also effective for removing a hydrophobic component produced by decomposition of protective groups in the resist upon exposure.

The saturated hydrocarbon compound obtained by the method of the present invention may be used as a mixture with a liquid other than the liquid of the present invention, as required, to adjust the optical property values such as the refractive index and the transmittance, and the property values such as the contact angle, specific heat, viscosity, and expansion coefficient to desired values.

As the liquid other than the liquid of present invention used for this purpose, a solvent which can be used for immersion exposure, an anti-foaming agent, a surfactant, and the like may be used. Use of such a liquid is effective for reducing bubbles or controlling surface tension.

A resist pattern can be formed by applying radiation to a photoresist film or a photoresist film on which an immersion upper layer film is formed using the saturated hydrocarbon compound of the present invention as a medium through a mask having a specific pattern, and then developing the photoresist film.

As the radiation used for immersion exposure, various types of radiation such as visible light; ultraviolet rays such as g-line and i-line; far ultraviolet rays such as an excimer laser light; X-rays such as synchrotron radiation; and charged particle rays such as electron beams may be selectively used depending on the photoresist film used and the combination of the photoresist film and the immersion upper layer film. In particular, it is preferable to use light from an ArF excimer laser (wavelength: 193 nm) or a KrF excimer laser (wavelength: 248 nm).

### EXAMPLES

The examples are given below. In the examples, absorbance was measured by collecting a liquid sample in a cell with a light path length of 1 cm having a polytetrafluoroethylene lid in a glove box in a nitrogen atmosphere controlled at 0.5 ppm or less using “JASCO-V-550” manufactured by JASCO Corporation, wherein the cell containing the above liquid was used as a sample and air was used as a reference. The absorbance is indicated by log₁₀(l₀/l), wherein l₀ is transmittance of the sample and l is transmittance of the reference.

The values shown in the table for each example were obtained by correcting reflection of the cell by calculation.

### Example 1

A raw material trans-decalin was produced by a refining process which comprises a first sulfuric acid washing treatment, second sulfuric acid washing treatment, and distillation under reduced pressure.

<First Sulfuric Acid Washing Treatment and Distillation Under Reduced Pressure>

In a nitrogen atmosphere, a 200 ml round bottom flask equipped with a glass-coated stirrer bar was charged with 100 ml of commercially available trans-decalin (manufactured by Tokyo Kasei Kogyo Co., Ltd.; absorbance at 193 nm wavelength converted to 1 cm optical path length is about 2.0). After the addition of 50 ml of concentrated sulfuric acid (manufactured by Wako Pure Chemical Industries, Ltd.), the mixture was stirred at 25° C. for 60 minutes at a stirrer bar rotational speed of 500 to 1,000 rpm. The concentrated sulfuric acid was then removed by separation. The separated organic layer was washed once with 50 ml of ultra-pure water and once with 50 ml of saturated aqueous solution of sodium hydrogencarbonate. The organic layer was then washed with 50 ml of ultra-pure water once. The above washing operation was carried out three times to confirm that the pH of the organic layer after washing with ultra-pure water was 7.0 (neutral). After drying the organic layer with magnesium sulfate, the magnesium sulfate was removed by decantation. The resulting product was then subjected to reduced pressure distillation under a pressure of 1 to 3 mmHg at 35 to 45° C. to obtain a total of 70 ml of refined trans-decalin. Absorbance per 1 cm of trans-decalin after refining was measured to find that the absorbance was 0.10.

<Second Sulfuric Acid Washing Treatment>

A 200 ml round bottom flask equipped with a glass-coated stirrer bar was charged with 70 ml of the refined trans-decalin (absorbance at 193 nm converted to 1 cm of optical path length: 0.10) obtained above. After the addition of 35 ml of concentrated sulfuric acid (manufactured by Aldrich Co., Ltd., purity: 99.999%), the mixture was stirred at 5° C. for 120 minutes at a stirrer bar rotational speed of 500 to 1,000 rpm. Absorbance per 1 cm of trans-decalin after refining was measured to find that the absorbance was 0.08.

<Distillation Under Reduced Pressure>

After the second sulfuric acid washing treatment, the trans-decalin was separated and put into a separate 200 ml round bottom flask, and subjected to reduced pressure distillation under a pressure of 1 to 3 mmHg at 35 to 45° C. to obtain a total of 15 ml of refined trans-decalin. Absorbance per 1 cm of trans-decalin after refining was measured to find that the absorbance was 0.06.

The total amount of sulfuric acid used was 185 ml.

### Comparative Example 1

In a nitrogen atmosphere, trans-decalin (manufactured by Tokyo Kasei Kogyo Co., Ltd.; absorbance at 193 nm wavelength converted to 1 cm optical path length: about 2.0) was subjected to refining treatment in the same manner as the second sulfuric acid washing treatment of Example 1. Absorbance per 1 cm of trans-decalin after refining was measured to find that the absorbance was 1.8.

### Comparative Example 2

The refined trans-decalin obtained in the first sulfuric acid washing treatment in Example 1 was subjected to refining treatment in the same manner as the second sulfuric acid washing treatment of Example 1, except that the stirring conditions were changed to a temperature of 25° C. and stirring duration for 60 minutes. Absorbance per 1 cm of trans-decalin after refining was measured to find that the absorbance was 0.10.

Comparison of Example 1 and Comparative Example 1 indicates that a saturated hydrocarbon compound with a low absorbance can be obtained by the second low-temperature sulfuric acid washing treatment when the absorbance before the treatment is 0.10 or less.

Comparison of Example 1 and Comparative Example 2 indicates that low-temperature sulfuric acid washing can remove impurities more efficiently than room-temperature sulfuric acid washing and can produce a liquid with a low absorbance.

### Example 2

<First Sulfuric Acid Washing Treatment>

In a nitrogen atmosphere, a 200 ml round bottom flask equipped with a glass-coated stirrer bar was charged with 100 ml of trans-decalin (manufactured by Tokyo Kasei Kogyo Co., Ltd.; absorbance at 193 nm wavelength converted to 1 cm optical path length is about 2.0). After the addition of 50 ml of concentrated sulfuric acid (manufactured by Aldrich Co., Ltd., purity: 99.999%), the mixture was stirred at 25° C. for 60 minutes at a stirrer bar rotational speed of 500 to 1,000 rpm. The trans-decalin was separated and put into a separate 200 ml round bottom flask equipped with a glass-coated stirrer bar, and the above procedure was carried out three times to obtain a total 70 ml of refined trans-decalin. Absorbance per 1 cm of trans-decalin after refining was measured to find that the absorbance was 0.14.

<Second Sulfuric Acid Washing Treatment and Distillation Under Reduced Pressure>

The refined trans-decalin obtained by the above experiment was subjected to refining treatment in the same manner as the second sulfuric acid washing treatment and distillation under reduced pressure of Example 1. Absorbance per 1 cm of trans-decalin after refining was measured to find that the absorbance was 0.07.

### Example 3

<First Sulfuric Acid Washing Treatment>

In a nitrogen atmosphere, a 200 ml round bottom flask equipped with a glass-coated stirrer bar was charged with 100 ml of trans-decalin (manufactured by Tokyo Kasei Kogyo Co., Ltd.; absorbance at 193 nm wavelength converted to 1 cm optical path length is about 2.0). After the addition of 50 ml of concentrated sulfuric acid (manufactured by Aldrich Co., Ltd., purity: 99.999%), the mixture was stirred at 25° C. for 60 minutes at a stirrer bar rotational speed of 500 to 1,000 rpm. The trans-decalin was separated and put into a separate 200 ml round bottom flask equipped with a glass-coated stirrer bar, and the above procedure was carried out two times to obtain a total 70 ml of refined trans-decalin. Absorbance per 1 cm of trans-decalin after refining was measured to find that the absorbance was 0.25.

<Second Sulfuric Acid Washing Treatment and Distillation Under Reduced Pressure>

The refined trans-decalin obtained by the above experiment was subjected to refining treatment in the same manner as the second sulfuric acid washing treatment and distillation under reduced pressure of Example 1. Absorbance per 1 cm of trans-decalin after refining was measured to find that the absorbance was 0.08.

### Example 4

<First Sulfuric Acid Washing Treatment>

In a nitrogen atmosphere, a 200 ml round bottom flask equipped with a glass-coated stirrer bar was charged with 100 ml of trans-decalin (manufactured by Tokyo Kasei Kogyo Co., Ltd.; absorbance at 193 nm wavelength converted to 1 cm optical path length is about 2.0). After the addition of 50 ml of concentrated sulfuric acid (manufactured by Aldrich Co., Ltd., purity: 99.999%), the mixture was stirred at 25° C. for 60 minutes at a stirrer bar rotational speed of 500 to 1,000 rpm. The trans-decalin was separated and put into a separate 200 ml round bottom flask. A total 70 ml of refined trans-decalin was thus obtained. Absorbance per 1 cm of trans-decalin after refining was measured to find that the absorbance was 0.37.

<Second Sulfuric Acid Washing Treatment and Distillation Under Reduced Pressure>

The refined trans-decalin obtained by the above experiment was subjected to refining treatment in the same manner as the second sulfuric acid washing treatment and distillation under reduced pressure of Example 1. Absorbance per 1 cm of trans-decalin after refining was measured to find that the absorbance was 0.09.

### Example 5

<First Sulfuric Acid Washing Treatment and Distillation Under Reduced Pressure>

In a nitrogen atmosphere, a 200 ml round bottom flask equipped with a glass-coated stirrer bar was charged with 100 ml of trans-decalin (manufactured by Tokyo Kasei Kogyo Co., Ltd.; absorbance at 193 nm wavelength converted to 1 cm optical path length is about 2.0). After the addition of 50 ml of concentrated sulfuric acid (manufactured by Aldrich Co., Ltd., purity: 99.999%), the mixture was stirred at 25° C. for 60 minutes at a stirrer bar rotational speed of 500 to 1,000 rpm. The trans-decalin was separated and put into a separate 200 ml round bottom flask and distilled under reduced pressure of 1 to 3 mmHg at 35 to 45° C.

<Second Sulfuric Acid Washing Treatment and Distillation Under Reduced Pressure>

The refined trans-decalin obtained above was added to 50 ml of concentrated sulfuric acid together with a glass-coated stirrer bar. The mixture was stirred at 5° C. for 120 minutes at a stirrer bar rotational speed of 500 to 1,000 rpm. After that, the trans-decalin was separated and put into a separate 200 ml round bottom flask, and subjected to reduced pressure distillation under a pressure of 1 to 3 mmHg at 35 to 45° C. to obtain a total 70 ml of refined trans-decalin. Absorbance per 1 cm of trans-decalin after refining was measured to find that the absorbance was 0.08.

### Example 6

<First Sulfuric Acid Washing Treatment>

In a nitrogen atmosphere, a 200 ml round bottom flask equipped with a glass-coated stirrer bar was charged with 100 ml of trans-decalin (manufactured by Tokyo Kasei Kogyo Co., Ltd.; absorbance at 193 nm wavelength converted to 1 cm optical path length is about 2.0). After the addition of 50 ml of concentrated sulfuric acid (manufactured by Aldrich Co., Ltd., purity: 99.999%), the mixture was stirred at 25° C. for 60 minutes at a stirrer bar rotational speed of 500 to 1,000 rpm.

<Second Sulfuric Acid Washing Treatment and Distillation Under Reduced Pressure>

After the first sulfuric acid washing treatment, the mixture was stirred at 5° C. for 120 minutes at a stirrer bar rotational speed of 500 to 1,000 rpm without changing the sulfuric acid used in the previous treatment. After that, the trans-decalin was separated and put into a separate 200 ml round bottom flask, and subjected to reduced pressure distillation under a pressure of 1 to 3 mmHg at 35 to 45° C. to obtain a total 70 ml of refined trans-decalin. Absorbance per 1 cm of trans-decalin after refining was measured to find that the absorbance was 0.10.

The total amount of sulfuric acid used was 50 ml.

### Example 7

<First Sulfuric Acid Washing Treatment>

In a nitrogen atmosphere, a 200 ml round bottom flask equipped with a glass-coated stirrer bar was charged with 100 ml of trans-decalin (manufactured by Tokyo Kasei Kogyo Co., Ltd.; absorbance at 193 nm wavelength converted to 1 cm optical path length is about 2.0). After the addition of 50 ml of concentrated sulfuric acid (manufactured by Aldrich Co., Ltd., purity: 99.999%), the mixture was stirred at 25° C. for 60 minutes at a stirrer bar rotational speed of 500 to 1,000 rpm.

<Second Sulfuric Acid Washing Treatment and Distillation Under Reduced Pressure>

After the first sulfuric acid washing treatment, the sulfuric acid used in the previous treatment was replaced with fresh sulfuric acid and the mixture was stirred at 5° C. for 120 minutes at a stirrer bar rotational speed of 500 to 1,000 rpm. After that, the trans-decalin was separated and put into a separate 200 ml round bottom flask, and subjected to reduced pressure distillation under a pressure of 1 to 3 mmHg at 35 to 45° C. to obtain a total 70 ml of refined trans-decalin. Absorbance per 1 cm of trans-decalin after refining was measured to find that the absorbance was 0.09.

The total amount of sulfuric acid used was 100 ml.

### Comparative Example 3

In a nitrogen atmosphere, a 200 ml round bottom flask equipped with a glass-coated stirrer bar was charged with 100 ml of trans-decalin (manufactured by Tokyo Kasei Kogyo Co., Ltd.; absorbance at 193 nm wavelength converted to 1 cm optical path length is about 2.0). After the addition of 50 ml of concentrated sulfuric acid (manufactured by Aldrich Co., Ltd., purity: 99.999%), the mixture was stirred at 25° C. for 60 minutes at a stirrer bar rotational speed of 500 to 1,000 rpm.

The trans-decalin was separated and put into a separate 200 ml round bottom flask equipped with a glass-coated stirrer bar, and the above procedure was carried out three times. After that, the trans-decalin was distilled under reduced pressure of 1 to 3 mmHg at 35 to 45° C. to obtain a total 70 ml of refined trans-decalin. Absorbance per 1 cm of trans-decalin after refining was measured to find that the absorbance was 0.10/cm.

The total amount of sulfuric acid used was 150 ml.

It can be understood from the comparison of Examples 6 and 7 with Comparative Example 3 that it is possible to reduce the amount of sulfuric acid used for the refining.

### Example 8

<First Sulfuric Acid Washing Treatment>

In a nitrogen atmosphere, a 200 ml round bottom flask equipped with a glass-coated stirrer bar was charged with 100 ml of trans-decalin (manufactured by Tokyo Kasei Kogyo Co., Ltd.; absorbance at 193 nm wavelength converted to 1 cm optical path length is about 2.0). After the addition of 50 ml of concentrated sulfuric acid (manufactured by Aldrich Co., Ltd., purity: 99.999%), the mixture was stirred at 25° C. for 60 minutes at a stirrer bar rotational speed of 500 to 1,000 rpm.

<Second Sulfuric Acid Washing Treatment and Distillation Under Reduced Pressure>

After the first sulfuric acid washing treatment, the mixture was stirred at 0° C. for 120 minutes at a stirrer bar rotational speed of 500 to 1,000 rpm without changing the sulfuric acid used in the previous treatment. After that, the trans-decalin was separated and put into a separate 200 ml round bottom flask, and subjected to reduced pressure distillation under a pressure of 1 to 3 mmHg at 35 to 45° C. to obtain a total 70 ml of refined trans-decalin. Absorbance per 1 cm of trans-decalin after refining was measured to find that the absorbance was 0.12.

### Example 9

<First Sulfuric Washing Acid Treatment>

In a nitrogen atmosphere, a 200 ml round bottom flask equipped with a glass-coated stirrer bar was charged with 100 ml of trans-decalin (manufactured by Tokyo Kasei Kogyo Co., Ltd.; absorbance at 193 nm wavelength converted to 1 cm optical path length is about 2.0). After the addition of 50 ml of concentrated sulfuric acid (manufactured by Aldrich Co., Ltd., purity: 99.999%), the mixture was stirred at 25° C. for 60 minutes at a stirrer bar rotational speed of 500 to 1,000 rpm.

<Second Sulfuric Acid Washing Treatment and Distillation Under Reduced Pressure>

After the first sulfuric acid washing treatment, the mixture was stirred at 0° C. for 120 minutes at a stirrer bar rotational speed of 500 to 1,000 rpm without changing the sulfuric acid used in the previous treatment. After that, the trans-decalin was separated and put into a separate 200 ml round bottom flask, and subjected to reduced pressure distillation under a pressure of 1 to 3 mmHg at 35 to 45° C. to obtain a total 70 ml of refined trans-decalin. Absorbance per 1 cm of trans-decalin after refining was measured to find that the absorbance was 0.10.

It can be understood from the comparison of Examples 6 and 8 with Example 9 that it is preferable to set the temperature for the second sulfuric acid treatment at 20° C. or more lower than the temperature for the first sulfuric acid treatment.

### INDUSTRIAL APPLICABILITY

According to the method for producing a saturated hydrocarbon compound of the present invention, it is possible to further reduce the absorbance of the saturated hydrocarbon compound especially with an absorbance of 0.10/cm or less at 193 nm by washing with sulfuric acid at a low temperature. Refining by washing with sulfuric acid at a low temperature can remove impurities more efficiently than refining at room temperature using a smaller amount of sulfuric acid. Therefore, the saturated hydrocarbon compound can be suitably used as a liquid for the immersion exposure method which is a technology essential for manufacturing semiconductor devices which will become more and more minute in the future.

1. A method for producing a saturated hydrocarbon compound comprising a sulfuric acid washing treatment to reduce absorbance,
wherein the sulfuric acid washing treatment comprises two or more sulfuric acid washing treatment steps, each conducted at a temperature differing from the other steps, at least the temperature in the last sulfuric acid washing treatment step being lower than the temperature in previous sulfuric acid washing treatment steps.

2. The method according to claim 1, wherein the temperature of the last sulfuric acid washing treatment step is from −40° C. to 10° C.

3. The method according to claim 1, wherein the two or more sulfuric acid washing treatment steps comprise a first sulfuric acid washing treatment step and a second sulfuric acid washing treatment step, the second sulfuric acid washing treatment step being conducted after the first sulfuric acid washing treatment step at a temperature 10° C. or more lower than the temperature of the first sulfuric acid washing treatment step.

4. The method according to claim 3, wherein the temperature difference between the first sulfuric acid washing treatment step and the second sulfuric acid washing treatment step is from 10° C. to 75° C.

5. The method according to claim 4, wherein the temperature difference is from 20° C. to 75° C.

6. The method according to claim 3, wherein the temperature of the second sulfuric acid washing treatment step is from −40° C. to 10° C.

7. The method according to claim 6, wherein the temperature of the first sulfuric acid washing treatment step is from 15° C. to 35° C.

8. The method according to claim 3, wherein the absorbance of light with a wavelength of 193 nm of the saturated hydrocarbon compound treated in the second sulfuric acid washing treatment step is 0.10 or less per 1 cm of optical path length of the liquid.

9. The method according to claim 3, wherein the same sulfuric acid is used in the first sulfuric acid washing treatment step and the second sulfuric acid washing treatment step.

10. The method according to claim 1, further comprising a distillation refining step after the last sulfuric acid washing treatment step.

11. The method according to claim 1, wherein the saturated hydrocarbon compound is an alicyclic saturated hydrocarbon compound.

12. The method according to claim 11, wherein the alicyclic saturated hydrocarbon compound is decalin.

13. A method for producing a saturated hydrocarbon compound comprising a sulfuric acid washing treatment to reduce absorbance,
wherein the sulruric acid washing treatment comprises two or more sulfuric acid washing treatment steps, each conducted at a temperature differing from the other steps, and the temperature of the last sulfuric acid washing treatment step is from −40° C. to 10° C.

14. The method according to claim 13, wherein the absorbance of light with a wavelength of 193 nm of the saturated hydrocarbon compound used as a raw material for the sulfuric acid washing treatment step is 0.10 or less per 1 cm of optical path length of the liquid.

15. The method according to claim 13, further comprising a distillation refining step after the sulfuric acid washing treatment steps.

16. The method according to claim 13, wherein the saturated hydrocarbon compound is an alicyclic saturated hydrocarbon compound.

17. The method according to claim 16, wherein the alicyclic saturated hydrocarbon compound is decalin.
